(19) United States
(12) Patent Application Publication
(10) Pub. No.: US 2022/0233413 A1
(43) Pub. Date: Jul. 28, 2022

(54) MONODISPERSE EMULSIONS TEMPLATED BY THREE-DIMENSIONAL STRUCTURED MICROPARTICLES AND METHODS OF MAKING THE SAME

(71) Applicant: THE REGENTS OF THE UNIVERSITY OF CALIFORNIA, Oakland, CA (US)

(72) Inventors: Dino Di Carlo, Los Angeles, CA (US); Chueh-Yu Wu, Los Angeles, CA (US); Andrea L. Bertozzi, Los Angeles, CA (US); Bao Wang, Los Angeles, CA (US); Joseph de Rutte, Los Angeles, CA (US); Kyung HA, Los Angeles, CA (US)

(73) Assignee: THE REGENTS OF THE UNIVERSITY OF CALIFORNIA, Oakland, CA (US)

(21) Appl. No.: 17/609,384

(22) PCT Filed: May 5, 2020
(86) PCT No.: PCT/US2020/031522

§ 371 Date: Nov. 6, 2021

Related U.S. Application Data
(60) Provisional application No. 62/844,391, filed on May 7, 2019.

(51) Int. Cl. A61K 8/06; A61K 8/02

(52) U.S. Cl. CPC A61K 8/06; A61K 8/0241; A61K 2800/10; A61K 2800/612; A61K 2800/412

(57) ABSTRACT

An emulsion system includes a plurality of monodisperse particle-drops. Each particle-drop is formed by a single elongated drop-carrier particle disposed in an oil-based continuous phase, wherein the single elongated drop-carrier particle comprises an elongate body with an opening at one end thereof. The single elongated drop-carrier particle has a hydrophilic interior region containing an aqueous droplet and a hydrophobic exterior region. The aqueous droplet and/or a surface of the hydrophilic interior region may contain one or more reagents, analytes, labels, reporter molecules, and/or cells.

### RELATED APPLICATION

This application claims priority to U.S. Provisional Patent Application No. 62/844,391 filed on May 7, 2019, which is hereby incorporated by reference in its entirety. Priority is claimed pursuant to 35 U.S.C. § 119 and any other applicable statute.

### STATEMENT REGARDING FEDERALLY SPONSORED RESEARCH AND DEVELOPMENT

This invention was made with government support under Grant Number GM126414, awarded by the National Institutes of Health. The government has certain rights in the invention.

### TECHNICAL FIELD

The technical field generally relates to monodisperse emulsions that are formed or templated by the user of three-dimensional (3D) structured microparticles that can have sculpted surface chemistries. The resulting emulsion ensemble generates uniformly sized and shaped droplets when simply mixed with two immiscible fluid phases.

### BACKGROUND

Emulsions provide significant value to products in the food, cosmetics, paints, oil and pharmaceutical industries, allowing the creation of stabilized liquids and materials from fluid components that normally possess incompatible chemical properties. Monodisperse emulsions of water in oil are also driving the ultimate limits of molecular and cellular analysis, leveraging reactions on single molecules and cells that proceed at similar rates across uniformly sized aqueous compartments and without cross-talk. However, emulsions are only metastable, requiring energy to create them and surface effects to help stabilize the interface between the two immiscible fluids. Coalescence of drops leads to thermodynamic equilibrium, resulting in non-uniform drop sizes that can change with temperature or time. Association of a single solid phase with each drop also enables surface-based reactions and barcoding, which has led to transformative applications in single-cell and single-molecule analysis and synthesis, but is usually limited by random encapsulation processes.

International Patent Application Publication No. WO 2018/156935 ('935 application), for example, discloses sub-millimeter scale three dimensional structures referred to as drop-carrier particles. The drop-carrier particles allow the selective association of one solution (i.e., a dispersed phased) with an interior region of each of the drop-carrier particles, while a second non-miscible solution (i.e., a continuous phase) associates with an exterior region of each of the drop-carrier particles due to the specific chemical and/or physical properties of the interior and exterior regions of the drop-carrier particles. The combined drop-carrier particle with the dispersed phase contained therein is referred to as a particle-drop. The selective association results in compartmentalization of the dispersed phase solution into sub-microliter-sized volumes contained in the drop-carrier particles. The compartmentalized volumes can be used for single-molecule assays as well as single-cell, and other single-entity assays. Improvements in drop-carrier particles and methods of use thereof are needed.

### SUMMARY

In one embodiment of the invention, particular three-dimensional (3D) structured microparticles are disclosed with sculpted surface chemistries (referred to herein as drop-carrier particles (DCPs)) that template uniformly-sized and shaped drop ensembles when mixed with two immiscible fluid phases. The resulting drop ensembles that are formed each include a single DCP having an aqueous fluid residing therein, with the DCP (which contains the aqueous fluid therein) further residing in a small volume of immiscible oil to form the final ensemble or what are called particle-drops. In contrast to traditional emulsions, the templated particle-drop ensembles are thermodynamically stabilized at a defined volume for structures in which the volume vs. interfacial energy curve transitions from concave to convex.

In one particular embodiment, amphiphilic 3D C-shaped drop-carrier particles are manufactured having an inward-facing hydrophilic surface (i.e., the inner surface of the “C” shape) and an outward-facing hydrophobic surface (i.e., the outer surface of the “C” shape). Upon mixing the drop-carrier particles with two immiscible fluid phases, the particle-drop system maintains a monodisperse state and has the ability to conduct solid phase reactions, wash, and exchange solutions (or prevent exchange), and digitize the capture of microparticles. Further, cells may be captured within the DCP (in the aqueous fluid) and maintain their viability. Cells that are captured within the DCP may also be subject to manipulation (e.g., encapsulation and lysis). The particle-drops may be readily formed without the need for specialized and expensive microfluidic devices and instruments to operate these devices. The particle-drops are thermodynamically stable over long periods of time and are compatible with living cells. In some embodiments, a solid or gel phase of the DCP may concentrate and retain fluorophores or fluorescent labels which can then be used in downstream analysis, such as the DCP subsequently run through a fluorescent activated cell sorter (FACS) system or other flow cytometer or imaging flow cytometer system.

The DCPs may be used to capture uniform sized nanoliter-scale aqueous droplets therein. The DCPs may include materials with tailored interfacial tensions: an inner hydrophilic layer and outer hydrophobic layer. Because the monodisperse condition is an equilibrium state, this “armored” emulsion can be produced all at once with simple mixing and centrifugation steps, without complex instrumentation or other devices. Each liquid drop or droplet is associated with a solid-phase particle (i.e., the drop-carrier particle) that imparts unique properties to the drops in the resulting emulsion (i.e., particle-drop).

The emulsion system or ensemble mixture may be generated by mixing, pipetting, or agitating a system that includes DCPs, an aqueous phase, and an immiscible phase. The emulsion system includes particle-drops and optionally satellite drops which may be optionally removed from the collection of particle-drops in some embodiments. While the liquid-liquid interface in the system consists of particle-drops and satellite drops, the former can be isolated using its unique properties from the latter. This separation may be needed in applications where the particle-drops are analyzed downstream. For particle-drops, one drop is associated with one particle with a solid phase that imparts unique properties to the drops in the emulsion, which is different from conventional emulsions consisting in multiple molecules (surfactant) or multiple nanoparticles (Pickering emulsion). A main advantage for this type of system over conventional emulsions is that manufacturing of drop-carrier particles can be centralized and distributed to end users which differs from the current method to generate monodisperse drops, where end users must purchase and operate complex equipment to operate microfluidic devices and perform liquid handling to create monodisperse drops. Particle-drops may be formed with one droplet per engineered particle by simple shaking and agitation using low cost bench top equipment. The drop-carrier particles may be manufactured elsewhere stored and used as needed at the point-of-use.

There is significant potential, across a range of fields, for the use of thermodynamically stabilized microdroplets associated with solid compartments. The ability for each compartment to be chemically modified with biomolecules such as, for instance, affinity ligands, nucleic acids, or sensing molecules is a key feature for future controlled biological reactions and barcoding. Because each particle-drop is associated with a chemically-defined compartment, and the compartment can be sized or dimensioned to hold only a single-cell or organism, limitations of Poisson loading of cells and beads in standard emulsions are overcome. Such systems enable single-molecule analysis and synthesis, or a way to barcode molecules for single-cell analysis. In addition, the digitized solid (or gel) structure provides a general substrate to store information from reactions or impart new physical properties into monodisperse emulsions, such as modifications in shape, buoyancy, stiffness, magnetic properties, or stimuli-responsiveness, enabling new opportunities for “lab-on-a-particle” technologies.

In one embodiment, an emulsion system includes a plurality of monodisperse particle-drops wherein each particle-drop is formed by a single elongated drop-carrier particle disposed in an oil-based continuous phase, wherein the single elongated drop-carrier particle comprises an elongate body with an opening at one end thereof, the single elongated drop-carrier particle having a hydrophilic interior region containing an aqueous droplet and a hydrophobic exterior region.

In another embodiment, a method of forming an emulsion system of particle-drops includes providing a plurality of drop-carrier particles includes an elongate body with an opening at one end thereof, wherein an interior surface of the elongate body is hydrophilic and an exterior surface of the elongate body is hydrophobic; providing an aqueous solution containing one or more reagents, analytes, labels, reporter molecules, beads, and/or cells therein; providing an oil-based continuous phase; and subjecting the plurality of drop-carrier particles, aqueous solution, and oil-based continuous phase to an agitation operation to generate particle-drops.

In another embodiment, a method of using an emulsion system includes forming a plurality of monodisperse particle-drops, wherein each of the plurality of particle-drops is formed by a single drop-carrier particle disposed in an oil-based continuous phase, wherein the single drop-carrier particle includes an elongate body with an opening at one end thereof and further has a hydrophilic interior region containing an aqueous droplet and a hydrophobic exterior region and wherein the single drop-carrier particle and/or the aqueous droplet contains one or more reagents, analytes, labels, reporter molecules, and/or cells therein; and analyzing the particle-drops or the drop-carrier particles based on light or other reporting signal emitted therefrom.

### BRIEF DESCRIPTION OF THE DRAWINGS

FIG. 1A illustrates a drop-carrier particle according to one embodiment.

FIG. 1B illustrates an example of a single particle-drop. The particle-drop is contained in an oil-based continuous phase with the drop-carrier particle 12 containing an aqueous fluid droplet therein.

FIG. 1C schematically illustrates the formation of monodisperse particle-drops by batch mixing and centrifugation operations. Drop-carrier particles are manufactured with poly (ethylene glycol) and poly (propylene glycol) as the hydrophilic and hydrophobic layers, respectively in this example. Particle-drops with aqueous solution containing fluorescent dye are shown in the bottom of a vial. Insets in the right circle show a single particle-drop in brightfield and FITC channels. All scale bars are 500 μm.

FIG. 2A illustrates a histogram of drop size for particle-drops and free drops formed with Pluronic. The engineered particle-drops in both toluene and PSDS continuous phases (i.e., oil phases) have a narrow distribution with a strong mode in the distribution at a diameter of ˜200 μm. An emulsion of free drops (stabilized by Pluronic and no DCPs) using the same two phases do not prescribe a preferred drop size. It is noted that for free drops there is no ethanol included in the disperse phase because an emulsion is not stabilized by Pluronic in a toluene-ethanol-water system.

FIG. 2B illustrates a histogram of a shape metric, i.e., circularity, for particle-drops and free drops with surfactant, showing that particle-drops also are stabilized with a unique non-spherical morphology defined by the engineered template.

FIG. 2C illustrates a histogram of nominal diameter for two different shaped DCPs (elongated DCPs with a small opening of ˜50 μm or shorter DCPs with large opening of 85 μm). Elongated DCPs are ˜600 μm in length while shorter DCPs are ˜350 μm in length. Both particle types have a width of ˜250 μm. The shape of the DCPs is important to determine the shape and monodispersity of particle-drops. The eccentricity of supported drops can be reduced by using shorter DCPs with a wider opening. However, more than one of these shorter, open DCPs can assemble around larger drops, leading to increased polydispersity, although monodispersity still remains higher than for free drops.

FIG. 2D illustrates images taken before and after mixing and agitation of two groups of particle-drops with either biotin-4-fluorescein (BF) (MW 644.7, green) or rhodamine B isothiocyanate dextran (RBD) (MW 70,000 Da, red). The green and red dyes do not transfer between particle-drops after loading. All scale bars are 200 μm.

FIG. 3A shows Volume vs. Energy (V-E) curves showing that free drops and surfactant stabilized drops in an immiscible solution possess monotonically increasing energies with V^{2/3}, resulting in a thermodynamic driving force for coalescence. Drop-carrier particles (solid line), however, possess an energy vs. volume curve with a local minimum when the drop-carrier particle is substantially filled with fluid (ivₘᵢₙ). Other configurations of filling are shown along the curve (i-vi). The energy and volume corresponding to the local minimum are defined as E₀ and V₀.

FIG. 3B shows the presence of an inflection point in the V-E curve leads to spontaneous splitting of a drop between two drop-carrier particles resulting in increased monodispersity. A drop with a volume at the local energy minimum (V_{N}=1) cannot split to decrease energy (dashed line, 0 and 1). For a drop of volume V_{N}=2, the energy is minimized when splitting the droplet in half (solid line, minimum at V₁/N_{N}=0.5) distributed between two carrier particles.

FIG. 3C shows a phase diagram illustrating the interaction of immiscible fluid drops with the drop-carrier particle as a function of contact angles with the hydrophobic and hydrophilic surfaces (θ₂ and θ₃) for a drop volume V₀. Sample configurations show complete filling (θ₂>90 and θ₃<90, dark grey), while outside of this region, complex non-filling configurations are observed.

FIG. 4A illustrates images of a particle-drop before and after enzymatic reactions occurring internal to the particle-drop. After incubation, FDG was converted into fluorescein and remained in the drop to yield higher intensity in the green fluorescent channel than before. This indicates that the enzyme remains active in the particle-drop, which is the key component of enzyme amplified immunoassays.

FIG. 4B illustrates an image and graph of the red fluorescent intensity of particle-drops after HRP (horseradish peroxidase)-catalyzed reactions of QuantaRed reagents to resorufin with different concentrations of HRP located on the inner surface layer of the DCPs. The intensity (AU) was increased significantly with increasing concentration of HRP.

FIG. 4C shows time-lapse images of microalgae within a particle-drop, showing cell viability and continued motility over two (2) days, indicating the biocompatibility of the continuous oil phase used.

FIG. 4D illustrates time-lapse images of the process of cell lysis in a particle-drop. Fluorescent calcein inside the cell encapsulated in a particle-drop was shown to be released into the particle-drop ˜9 minutes after PSDS with sarkosyl was applied to the continuous phase around the particle-drop. This indicates that the sarkosyl detergent transferred into the aqueous phase from the oil phase and created pores in the cell membrane leading to lysis. All scale bars are 200 μm.

FIG. 5 illustrates how DCPs are formed according to one embodiment. Polymer precursors of poly (ethylene glycol) diacrylate (PEGDA) and poly (propylene glycol) diacrylate (PPGDA) are co-flowed with and without photoinitiator. The co-flowing streams are shaped using inertial flow sculpting to create a concentric C-shaped structure in the cross-section of the flow with PEGDA internal to PPGDA. Flow control of syringe pumps and the pinch valve is performed using a computer as illustrated. The sculpted flow is quickly stopped by use of a pinch valve which equalizes pressure at the inlet (Pᵢₙ) and outlet (Pₒᵤₜ) to P_{eq}. The sculpted stream is exposed to ultraviolet (UV) light to polymerize the PEGDA and PPGDA regions mixed with photoinitiator. The valve is opened and polymerized particles are collected before the cycle is repeated.

FIG. 6 illustrates the long-term stability of particle-drops. The particle-drops with fluorescein-containing aqueous solution were generated and imaged on day zero and day three. The size distribution of particle-drops over three days remains stable. The scale bar is 200 μm.

FIG. 7 illustrates the effect of aqueous volume on particle-drop formation. When the volume of the aqueous phase is too low it affects the distribution of nominal diameters among particle-drops. A number of incompletely filled particle-drops are seen for aqueous volumes of 10 μL (˜9 times the holding volume of the particles). The distribution of nominal diameter appears to saturate with a mode at 200 μm once the aqueous fluid reaches 20 μL (˜17 fold of the total holding volume of the particles).

FIG. 8 illustrates images showing the inhibition of solution exchange between particle-drops before and after agitation. Zoomed in regions are shown on top. Particle-drops were generated with 10 μg/mL biotin-4-fluorescein (BF) and 1 mg/mL rhodamine B isothiocyanate dextran (RBD) separately in two vials. ˜0.5 mL of particle-drop-laden solution from each vial were introduced into a new vial. The blended particle-drops in both FITC and TRITC channels were imaged before and after shaking the vial on a standard analog shaker (VWR, LLC.) for 4 minutes. Before and after agitation, only green and red fluorescent drops were observed, indicating there was no transport of dye between solid boundary-protected particle-drops. The scale bars in the top and bottom rows are 500 and 1000 μm respectively.

FIG. 9 illustrates solution exchange between particle-drops and satellite drops. Particle-drops were generated with 10 μg/mL BF and were mixed with a larger satellite drop with 1 mg/mL rhodamine B isothiocyanate dextran (RBD). The volume of the satellite drop is ˜20 times the total holding volume of the particle-drops. After agitation, red dye (RBD) was discovered in some particle-drops, showing that transport of molecules can be achieved when introducing non-protected aqueous drops of sufficient volume. The scale bars in the top and bottom rows are 200 and 500 μm respectively.

FIGS. 10A and 10B illustrates how free drops and surfactant-coated drops coalesce at thermodynamic equilibrium. The energy for partitioning one large free drop with volume, V_{N}, into two small drops with volumes (V₁, V₂), with (FIG. 10A) or without (FIG. 10B) surfactant, based on the V-E curves (i.e., E scaling with V^{2/3}) in FIG. 2A. The results show the drop energetically prefers to remain as a single drop in both cases (minima at 0 and 1).

FIGS. 11A-11C illustrate exemplary volume energy (V-E) curves and corresponding volume splitting plots (right-hand side). (FIG. 11A) For concave V-E curves (e.g., a spherical droplet) it is energetically favorable for volumes to coalesce into a single volume in order to minimize surface area. (FIG. 11B) For convex V-E curves it is energetically more favorable for a volume to split into equal volumes. This case also results in no preferred drop volume. (FIG. 11C) For the case of a V-E curve that transitions from convex to concave, there is an initial volume regime over which droplets split evenly (similar to the purely convex case). However, once the volume reaches twice the volume of the inflection point (V=2V₁=7.5), volumes split asymmetrically. Here the total volume splits into two volumes, a preferred smaller volume occurring over a large range of total volumes, and a larger volume containing the remaining volume.

The C-shaped drop-carrier particle design disclosed herein possesses these features, and supports monodisperse emulsions.

FIG. 12A illustrates system energy of a drop confined by parallel plates transitioning from a spherical cap to catenoid. The behavior of an aqueous drop with increasing volume is shown using a simplified model of two parallel plates confining the drop with a hydrophilic inner-facing layer. There is a change in morphology of the drop at equilibrium from a spherical cap to a catenoid bridging between the surface, which leads to a change in slope of the V-E curve, however, both remain concave. Additional features in the V-E curve (e.g., a convex initial region), and structures interacting with the drop, are needed to support monodisperse drops.

FIGS. 12B and 12C illustrate particle-drop morphologies as a function of aqueous volume (FIG. 12B) and time (FIG. 12C). FIG. 12B illustrates aqueous volumes (blue dye) from different minimal surface shapes when interacting with centimeter-sized DCPs depending on filling volume. The four types of morphologies shown in the experiments: spherical cap, bridge (catenoid), partial filling, and complete filling, agree well with numerical model predictions for (i) to (iv) in FIG. 2A. The scaled-up DCPs were fabricated using two PDMS molds replicated from two 3D printed structures, where one has the dimensions of the inner PEG layer, and the other has the size of the entire DCP. The inner PEG layer was crosslinked with yellow food dye in the 1^{st} mold, transferred it to the 2^{nd} mold to incorporate the PPG layer. The dash contour in the inset on the left-hand side represents the edge of the DCP. The Bond number of the scaled-up experiment is matched to that of the microscale particles by tuning solution densities. FIG. 12C illustrates time-lapse images of the development of a particle-drop leading to complete filling (iv). All scale bars are 5 mm and T is 0.47 second.

FIG. 13 illustrates images of monodisperse particle-drops. Drop-carrier particles, aqueous solution containing FITC-dextran, and oil phases were simply mixed in a scintillation vial and centrifuged down to generate particle-drops. The top row insets show stitched brightfield and fluorescence images of the entire vial generated using a microscopy. The white squares are magnified in FIG. 13. The bottom image is from a conventional camera with high magnification. The top and bottom scale bars are 1 and 2 mm respectively.

FIGS. 14A and 14B illustrates images of spherical microgels encapsulated in particle-drops. Gels loaded in the particle-drops exhibit loading following the Poisson distribution (inset graph FIG. 14A, histogram is experimental results, line is Poisson distribution). Isolation statistics are independent of size provided the gel is smaller than the drop-carrier particle opening. Loading with distributions that do not follow Poisson statistics can be achieved because of the steric interaction of the particles with the surrounding DCP. This can allow loading for example only a single hydrogel particle or cell per particle-drop if the DCP cavity is dimensioned to match that of the cell or particle. Multiple cells or particles can also be loaded if the cavity is dimensioned to hold the multiple cells or particles.

### DETAILED DESCRIPTION OF THE ILLUSTRATED EMBODIMENTS

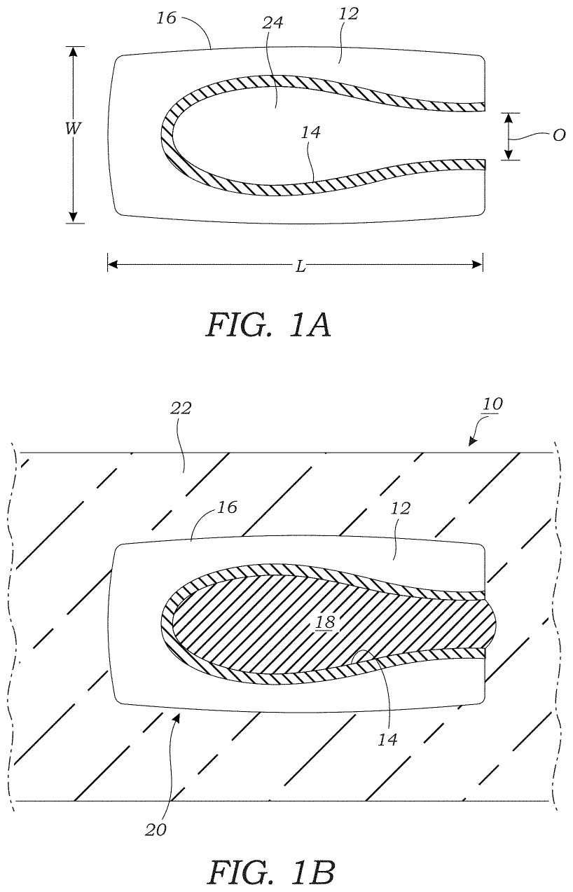

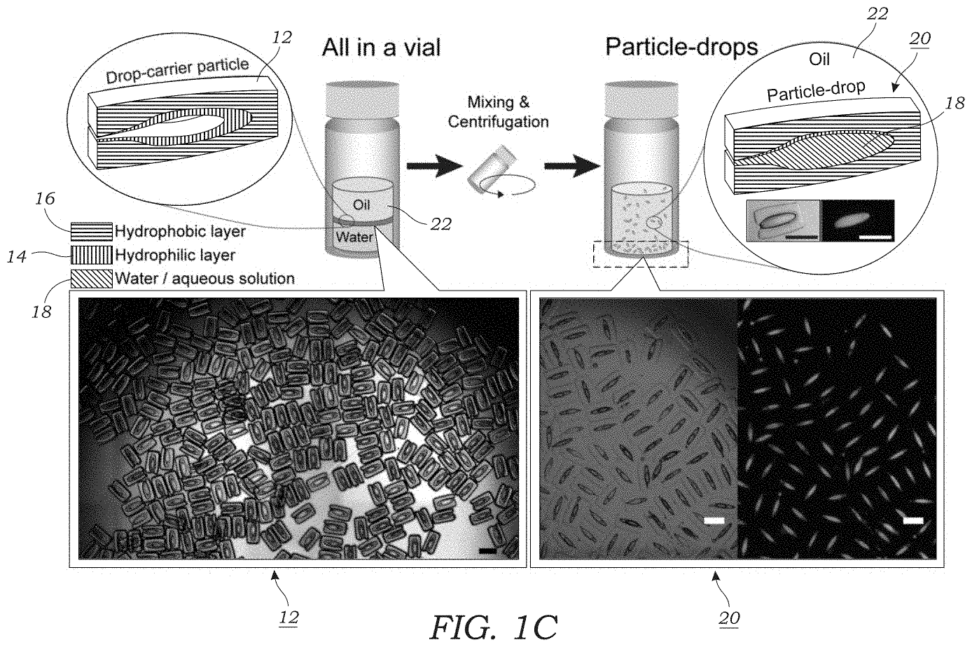

FIGS. 1A-1C illustrates one embodiment of a particle-drop system 10 and constituents thereof. The particle-drop system 10 includes a plurality of three-dimensional drop-carrier particles 12 (plurality is seen in FIG. 1C). The drop-carrier particles 12 are small, sub-millimeter scale solid particles that are formed having a particular geometric shape and have an interior region 14 and an exterior region 16. The interior region 14 of the drop-carrier particle 12 defines a three-dimensional volume that holds a fluid droplet 18. The fluid droplet 18 is the dispersed phase of an emulsion and, in one preferred embodiment, is an aqueous phase (e.g., formed from water). The interior region 14 of the drop-carrier particle 12 is, in one embodiment, hydrophilic. That is to say, the inner surface of the drop-carrier particle in this interior region 14 is hydrophilic. The hydrophilic nature of the interior region 14 may be achieved by the choice of material used during the manufacturing process used to make the drop-carrier particles 12 as explained herein. Alternatively, the interior region 14 may be rendered hydrophilic after formation of the drop-carrier particle 12. The exterior region 16 of the drop-carrier particle 12 is, in one embodiment, hydrophobic. That is to say, the exterior surface of the drop-carrier particle 12 in this exterior region 16 is hydrophilic. In another embodiment, the exterior region 16 of the drop-carrier particle 12 is fluorophilic (e.g., the exterior surface of the drop-carrier particle 12 in this exterior region 16 is fluorophilic). The hydrophobic or fluorophilic nature of the exterior region 16 may be achieved by the choice of material used during the manufacturing process used to make the drop-carrier particles 12 as explained herein. Alternatively, the exterior region 16 may be selectively rendered hydrophobic (or fluorophilic) after formation of the drop-carrier particle 12.

With reference to FIG. 1B, when the drop-carrier particle 12 is loaded with the droplet 18, the resulting construct is referred to herein as a particle-drop 20. A plurality of these particle-drops 20 form an emulsion which may be held within a container 21 such as a vial as seen in FIG. 1C. The container 21 may initially hold the drop-carrier particles 12 along with an aqueous solution and an oil-based solution. The container 21 or the contents thereof are then subject to an agitation process to form a plurality of particle-drops 20 that are disposed in oil 22 to form a particle-drop 20 emulsion (see right side of FIG. 1C). The oil 22 acts as the continuous phase while the aqueous-based droplet 18 acts as the dispersed phase. The oil 22 surrounds the particle-drops 20 to create a monodisperse particle-drop 20 emulsion. Monodisperse refers to the ability of the particle-drops 20 to retain substantially the same volume of fluid in each particle-drop 20. That is to say the volume of the fluid droplet 18 within each drop-carrier particle 12 is substantially the same across all particle-drops 20.

The monodisperse particle-drop 20 emulsions are created without the need of any complex or expensive instruments. Notably, the assembly of drop-carrier particles 12 supports a unique volume of an aqueous droplet 18, unlike droplets of multiple volumes supported by Pickering emulsions, such that a plurality of particle-drops 20 enables the formation of a monodisperse emulsion. As explained herein, drop-carrier particles 12 are formed from multiple material types into shaped particles with wetting surfaces that are strategically located, in some embodiments, on the interior of the drop-carrier particles 12. For example, hydrophilic material is polymerized or crosslinked using light exposure on the interior cavity of the drop-carrier particle 12 to form a hydrophilic surface while a separate hydrophobic material also polymerized or crosslinked using light surrounds the cavity or void 24 as is illustrated in FIG. 1A and forms a hydrophobic surface. In one preferred embodiment, the cavity or void 24 is open to the external environment of the drop-carrier particle 12 (i.e., there is one or more openings (O) that communicate with the external environment of the drop-carrier particle 12). The drop-carrier particles 12 may be made from known polymer materials that can be polymerized or crosslinked using, for example, light-initiated polymerization as explained herein.

The drop-carrier particles 12 that are used to form the particle-drops 20 are sub-millimeter sized particles in their longest dimension. Typically, the drop-carrier particles 12 have a longest dimension on the order of around 100-800 microns, although it should be appreciated that drop-carrier particles 12 of different sizes outside this specific range may also be used. In one preferred embodiment, the drop-carrier particle 12 has an elongate body as illustrated in FIGS. 1A and 1B. The length (L) of the drop-carrier particle 12 may be within the range of around 100-800 microns while the width (W) is typically less than half the length or around 50-400 microns.

As explained herein, in one preferred embodiment the drop-carrier particle 12 is elongated with a “C” shape. The C-shaped drop-carrier particle 12 has an opening (O) at one end thereof that provides access to the hydrophilic interior region (FIG. 1A). In some embodiments, the opening (O) has a dimension that is less than 100 μm. For example, this may include opening dimensions less than, 100 μm, 90 μm, 80 μm, 70 μm, 60 μm, 50 μm, 40 μm, 30 μm, and the like. As explained herein, a smaller opening in the drop-carrier particle generally yields greater monodispersity among the particle-drops.

In some embodiments, the surrounding oil-based continuous phase 22 is made of toluene, decanol, or other organic solvent (or mixtures thereof). In other embodiments, for example, where cells are contained in the drop-carrier particle 12, the surrounding oil-based continuous phase 22 may include poly(dimethylsiloxane-co-diphenylsiloxane) (PSDS). In some embodiments, the hydrophilic interior region includes a poly(ethylene glycol)-based material. This may include, for example, poly(ethylene glycol) diacrylate (PEGDA). The exterior region of the drop-carrier particle may be formed from poly(propylene glycol) diacrylate (PPGDA). In some embodiments, reaction products or chemical species or agents generated within the aqueous fluid droplets 18 located within the cavity or void 24 may migrate or partition into the material making up the hydrophilic interior region 14 of the drop-carrier particle 12 and be retained there for an extended period of time (e.g., several minutes, hours, or days). For example, fluorophores may be retained within the hydrophilic interior region 14 and remain there even despite washing of the drop-carrier particle 12. These drop-carrier particles 12 may then be analyzed and/or sorted using a device such as a FACS device.

To form the emulsion system a plurality of drop-carrier particles 12 are provided or otherwise obtained. These could be manufactured on-site or they could be manufactured elsewhere and stored for later use. Each of the plurality of drop-carrier particles 12 has a hydrophilic interior region 14 and a hydrophobic exterior region 16. An aqueous solution is provided that contains one or more reagents, chemical agents, analytes, labels, reporter molecules, beads, and/or cells therein. An oil-based continuous phase 22 is provided and the mixture of the plurality of drop-carrier particles 12, aqueous solution, and oil-based continuous phase 22 is then subject to an agitation operation. This may include, for example, shaking, pipetting (back-and-forth), and like. Optionally, centrifugation may be used to collect the formed emulsions. There are some droplets that are formed termed “satellite” drops that do not contain drop-carrier particles 12. These satellite drops may optionally be removed from the other particle-drops 20. These can be removed manually due to their buoyancy being different from particle-drops 20 (e.g., satellite drops generally are more buoyant and rise within the fluid while particle-drops 20 generally are less buoyant and remain at the bottom of the container or vessel containing the same). A filtration operation may also be performed to isolate the particle-drops 20 from the satellite drops.

In some embodiments, the hydrophilic interior region 14 contain one or more chemical species or moieties that are immobilized to a surface thereof. This provides the opportunity for such chemical species or moieties to participate in or facilitate various chemical and/or biological reactions. For example, one or more enzymes may be immobilized to the hydrophilic interior region (either directly or through a linker molecule or molecules). Likewise, various reporter molecules (e.g., fluorophores or other label) may also be immobilized to the hydrophilic interior region. Additional examples of species and objects that may be immobilized to the hydrophilic interior region include reagents, analytes, beads, and/or cells. In other embodiments, the chemical species or other objects may freely float or reside within the aqueous-based droplet 18. This may include one or more reagents, analytes, labels, reporter molecules, beads, and/or cells.

In one preferred embodiment, a single, live or living cell is located within the aqueous-based droplet 18. The cell may be a eukaryotic or prokaryotic cell. A single multi-cellular organism may also be loaded into the aqueous-based droplet 18. In still another alternative, a single microgel or bead may be loaded into the aqueous-based droplet 18. In other embodiments, multiple objects (e.g., cells, organisms, beads, microgels) may be loaded into the droplet 18. This may be accomplished by increasing the size of the void or cavity 24 in the drop-carrier particles 12. In one particular preferred embodiment, reagents may be exchanged through the oil-based continuous phase 22 and into the aqueous-based droplet 18. The ability to exchange reagents through the oil continuous phase 22 that can then partition into the aqueous phase of the droplet 18 of the particle-drops 20 can be used for other applications in performing bioassays, such as providing fluorogenic substrates for enzymatic reactions. In order to achieve reagent exchange through the oil phase 22 the reagent that is desired to exchange should be miscible in both the surrounding oil continuous phase 22 and disperse aqueous phase of the droplet 18. Preferably, the miscibility in the aqueous phase of the droplet 18 is higher than in the continuous surrounding phase 22 such that the reagent accumulates in the particle-drops 20 at a higher concentration. If the reagent is consumed in a reaction in the particle-drop 20 (e.g., for a fluorogenic substrate converted to a fluorophore, or a surfactant inserting into cell membranes) this can drive continued partitioning of the reagent into the particle-drop 20 from a large reservoir of reagent in the surrounding oil phase 22. This is important to transfer high quantities of reagent into the particle-drop 20 even if the saturation miscibility of the continuous oil phase may be at low concentrations.

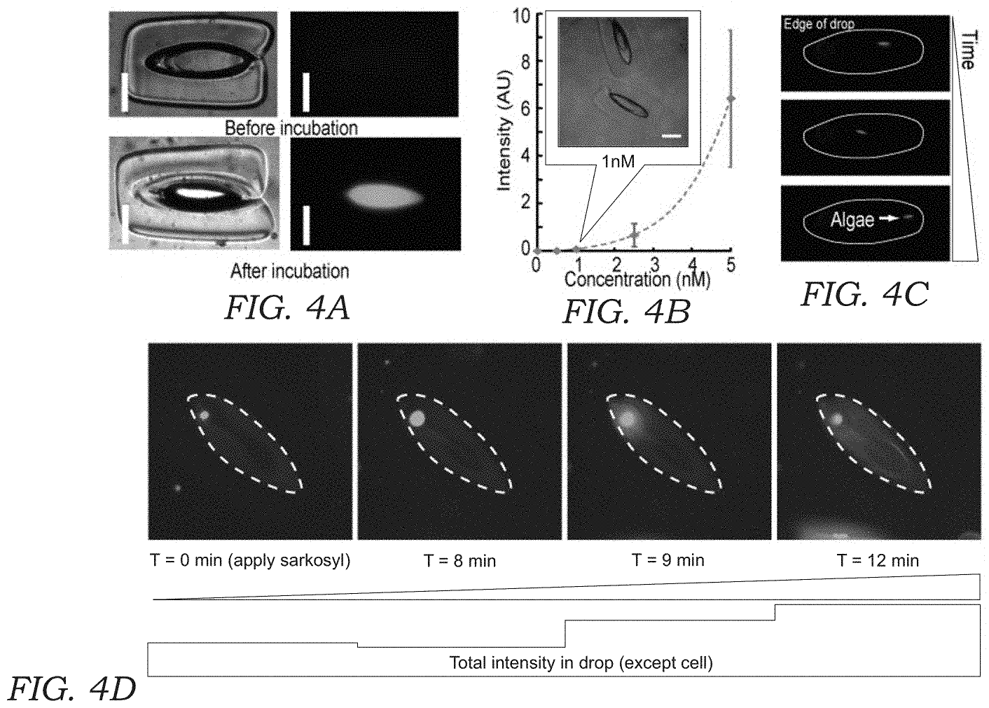

For example, a lysing agent in the form of a surfactant or detergent may be delivered from the surrounding oil phase 22 to the aqueous phase of the droplet 18 which can then causes lysis of the cell contained in the droplet 18 in the particle-drop 20. Lysis reagents such as sodium lauroyl sarcosinate or sarkosyl can be exchanged from the surrounding oil phase 22 to the aqueous phase of the droplet 18 which causes lysis of a cell contained in the particle-drop 20 (see FIG. 4D).

There is significant potential, across a range of fields, for the use of thermodynamically stabilized microdroplets (e.g., particle-drops 20) associated with solid compartments (drop-carrier particles 12). The ability for each compartment or region to be chemically modified with affinity ligands, nucleic acids, or sensing molecules is a key feature for future controlled biological reactions and barcoding. Because each particle-drop 20 is associated with a chemically-defined compartment, and the compartment can be sized to hold only a single-cell, limitations of Poisson loading of cells and beads in standard emulsions are overcome. Such systems enable single-molecule analysis and synthesis, or a way to barcode molecules for single-cell analysis. The digitized solid structure provides a general substrate to store information from reactions or impart new physical properties into monodisperse emulsions, such as modifications in shape, buoyancy, stiffness, magnetic properties, or stimuli-responsiveness, enabling new opportunities for “lab-on-a-particle” technologies.

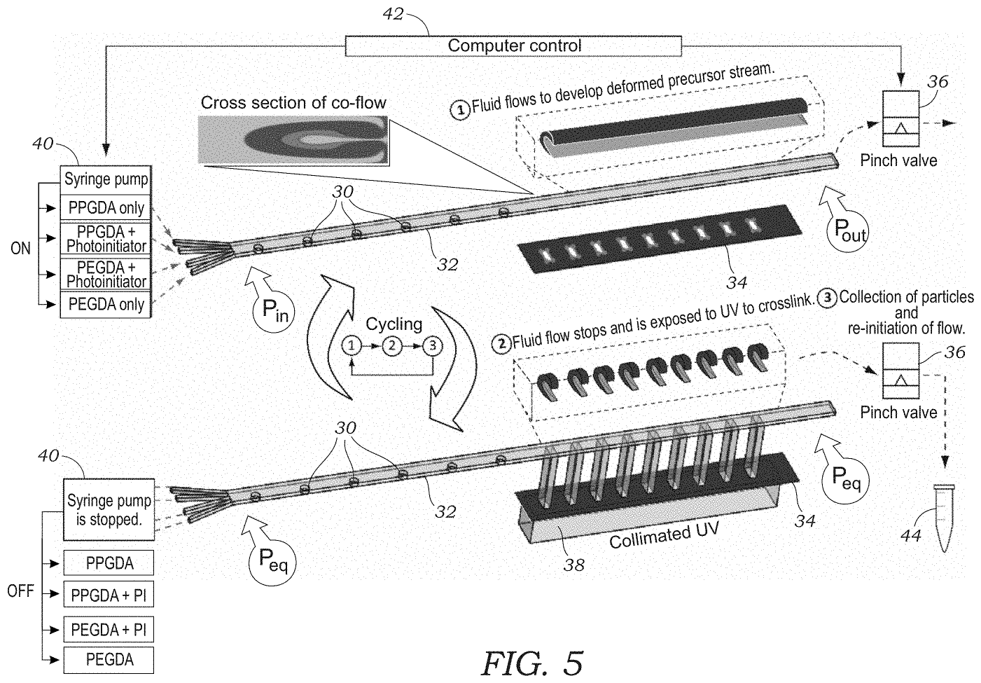

The drop-carrier particles 12 described herein can be manufactured using a known fabrication method called high-throughput Optical Transient Liquid Molding (OTLM). In this method and with reference to FIG. 5, microfluidic posts, pillars, or other protuberances 30 are formed in a microfluidic channel 32 and used to generate complex sub-millimeter scale drop-carrier particles 12 with shapes that consist of the orthogonal intersection of horizontally and vertically-extruded 2D patterns in a high-speed manner. An example of OTLM particle fabrication techniques is found in International Patent Application Publication No. WO/2017059367, which is incorporated herein by reference.

The horizontally and vertically-extruded 2D patterns (see operation #1 in FIG. 5 to create shaped precursor stream) are respectively determined by the cross-sectional shape of a flowstream of photo-crosslinkable polymer pre-cursor and the shape of an optical mask 34 that is used to generate the other orthogonal cross-section (see operation #2 in FIG. 5). Inertial flow engineering is used to sculpt a single-phase flow stream into a complex cross-sectional shape in the microfluidic channel 32 using the flow past a sequence of defined microstructures 40. The shape of the sculpted flow (a C-shaped precursor stream is seen in FIG. 5) may be user-defined and programmed using software to define the microfluidic channel 32 with the particular micropillar sequence necessary to create the final shape. For example, Wu et al., which is incorporated by reference herein, describe a software uFlow (available at http://biomicrofluidics.com/software.php) that allows for the design of 2D flow shapes with a simple graphical user interface (GUI) that can be used to predict and design particle shapes. See Wu et al., Rapid Software-Based Design and Optical Transient Liquid Molding of Microparticles, Adv. Materials, 27, pp. 7970-78 (2015).

Flowing through this microstructured channel creates a sculpted flow stream. The flow is then stopped using a pinch valve 36 and the stream is illuminated using patterned UV light from light source 38 through an optical mask 34 to achieve complex 3D drop-carrier particles 12. Automated control of the syringe pumps 40 and pinch valves 36 using computer 42 allows for a high production rate of tens of thousands of drop-carrier particles 12 per hour. In one embodiment, the inner hydrophilic region 14 that holds a liquid compartment for the droplet 18 is formed in the flow stream by deforming a precursor co-flow with hydrophilic and hydrophobic polymer precursors that are flowing side by side into a curved or encapsulated shape with concentric regions consisting of an interior void, hydrophilic, and hydrophobic layers. The orthogonal UV exposure pattern with cross-sectional shapes, such as optionally protruding shapes is designed to avoid the aggregation of drop-carrier particles 12. This pattern is exposed through a mask which contains the repeating pattern in a row along the flow direction to make many identical drop-carrier particles 12. The drop-carrier particles 12 can then be collected in vial or container 44.

While the embodiments described herein largely describe drop-carrier particles 12 having a hydrophilic interior region 14 and a hydrophobic exterior region 16, it should be appreciated that these regions could be reversed with the interior region 14 being hydrophobic (or fluorophilic) and the exterior region 16 being hydrophilic. In such an embodiment, the fluid droplet 18 that is carried by the drop-carrier particle 12 would be a hydrophobic fluid such as oil while the continuous phase that surrounds the particle-drops 20 would be an aqueous solution.

Experimental

Results:

DCPs 12 are manufactured by co-flowing pre-polymer solutions that are then crosslinked as seen in FIG. 5. The particle shape is sculpted along one direction using inertial fluid effects and in an orthogonal direction using photolithographic processes. Poly(ethylene glycol) diacrylate (PEGDA) and poly(propylene glycol) diacrylate (PPGDA), which are miscible with each other when suspended in ethanol, are the hydrophilic and hydrophobic polymer precursors. They are biocompatible with highly tunable physical and chemical properties.

A number of oil-based continuous phases were found to be immiscible with aqueous solution and are able to generate particle-drops 20, including toluene, Poly(dimethylsiloxane-co-diphenylsiloxane) (PSDS), decanol, and polypropylene glycol (e.g., uncrosslinked PPGDA). Toluene and PSDS were chosen as an oil phase 22 for experiments described herein because they are practical to handle with low viscosity and biocompatibility respectively. Depending on the liquid properties of continuous (oil) phases 22, protocols were developed for efficient particle-drop 20 generation. For low viscosity continuous phases 20 (e.g. toluene), DCPs 12 were dispersed in the continuous phase 22 first and then added to a controlled volume of the aqueous phase. For high viscosity continuous phases 22, DCPs 12 were resuspended in the aqueous phase with surfactant (e.g., Pluronic to avoid aggregation) to ensure there was enough interactions between DCPs 12 and the aqueous phase first. Particle-drop 20 generation was insensitive to the process, e.g., pipetting or centrifugation, and dispersion of DCPs 12 in the aqueous or continuous phase first. A number of continuous oil phases 22 were used to characterize particle-drops 20 and the capability to carry out applications requiring biocompatibility.

The protocol for producing particle-drops 20 from DCPs 12 is as follows. Manufactured DCPs 12 were purified using solvent (e.g., ethanol) and then transferred into an oil or water phase. The particle-laden oil (or water) solution was then mixed with a water (or oil) solution while controlling the volume ratio, and finally the mixed solution was centrifuged or pipetted in a glass vial to create particle-drops 20 (FIG. 1C). Particle-drops 20 in toluene and PSDS were characterized in terms of monodispersity and monomorphology.

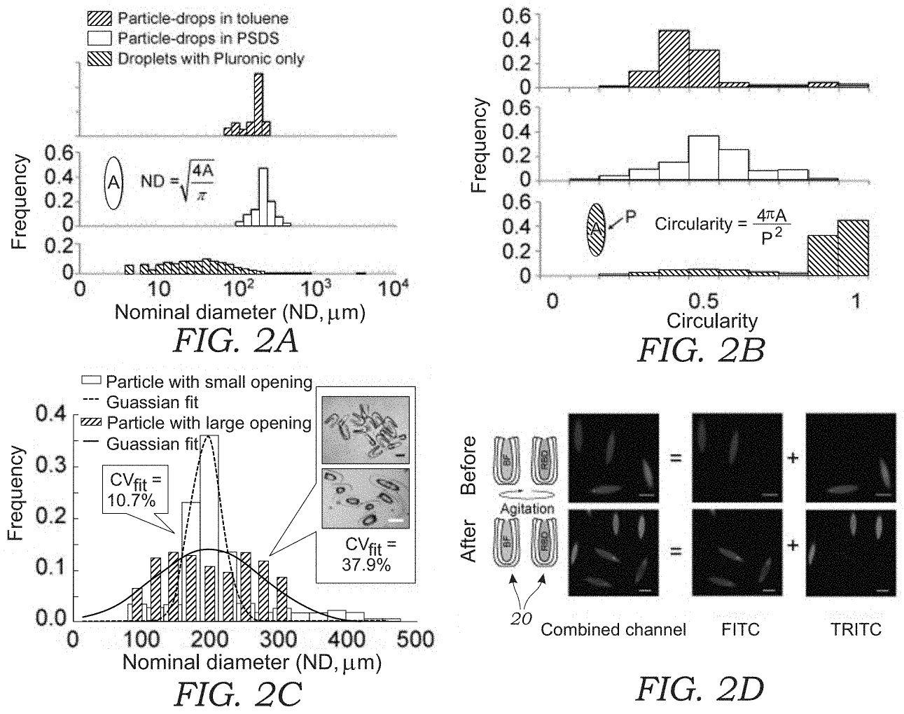

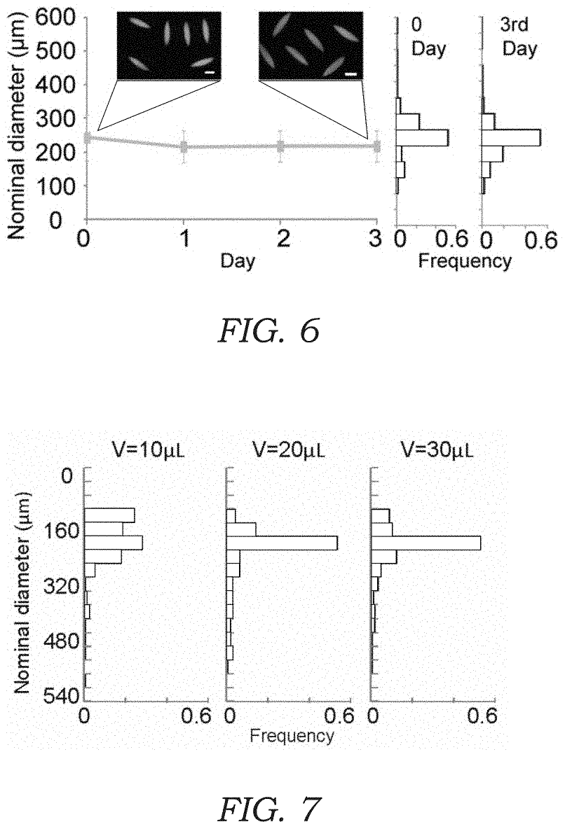

Monodispersity. The resulting particle-drops 20 in both toluene and PSDS have a preferred drop volume (nominal diameter of ˜200 μm) with reduced sensitivity to preparation conditions (FIG. 2A). Both continuous phases 22 show monodisperse emulsion formation with small coefficient of variations (CV) similar to microfluidic production of droplets, usually less than 10% or in other embodiments less than 40%. These CVs are less than what is observed for random emulsions formed by agitation or mixing (CVs>40%). In addition, a high percentage of the particle-drops 20 (>30 and >40%) occupied the mode of the distribution in the nominal diameter. The microstructure of the surrounding DCPs 12 not only templates the drops 18 in the emulsion but also sustains their shape over a long period of time, resisting the usual coarsening process found in standard spherical drop emulsions. Once created, the particle-drops 20 in toluene maintain the same mean volume and polydispersity index (PDI) for at least 3 days as long as the dispersed phase was prevented from evaporating (FIG. 6).

Monomorphology. The distribution of particle-drops 20 in toluene and PSDS is shown in (FIG. 2B), showing a sharp contrast between a standard emulsion (without DCP templating of drops) and the engineered particle-drop 20 system. The shape of particle-drops 20 is controllable and determined by the void space 24 of the DCPs 12 while surfactant-stabilized drops are always spherical. The comparison between toluene and PSDS shows that the particle-drops 20 in toluene adapt to the shape of the cavity within the DCPs 12 slightly more than in PSDS. Particle-drops 20 in PSDS slightly bulged out of the cavity, while still maintaining high uniformity in volume and shape. This result follows differences in interfacial tension of PSDS compared to toluene.

Particle-shape-dependent monodispersity. Drop-carrier particle 12 shape was shown to affect monodispersity. The monodispersity of an armored emulsion was found to depend on the detailed geometry of the drop-carrier particles 12. Higher aspect ratio drop-carrier particles 12 with a small opening (<50 μm, N=637) had a tighter distribution and well-defined mode in droplet nominal diameter (ND). However, shorter aspect ratio drop-carrier particles 12 with a wider opening (85 μm, N=185) had almost 4-fold higher variation in size. Qualitatively the larger opening was observed to allow two or more drop-carrier particles 12 to assemble around a single droplet (inset) in a more stable configuration, leading to more variation in drop sizes.

Filling. Drop diameter and morphology are also affected by the total volume of the aqueous phase in the experiment. When that volume is less than a saturation value, 20 μL, ˜20 fold of the entire void volume of the drop-carrier particles 12, a high percentage of the population was only partially filled with the aqueous phase (FIG. 7). Once filled, a strong mode in the distribution of nominal diameter is observed at ˜200 μm.

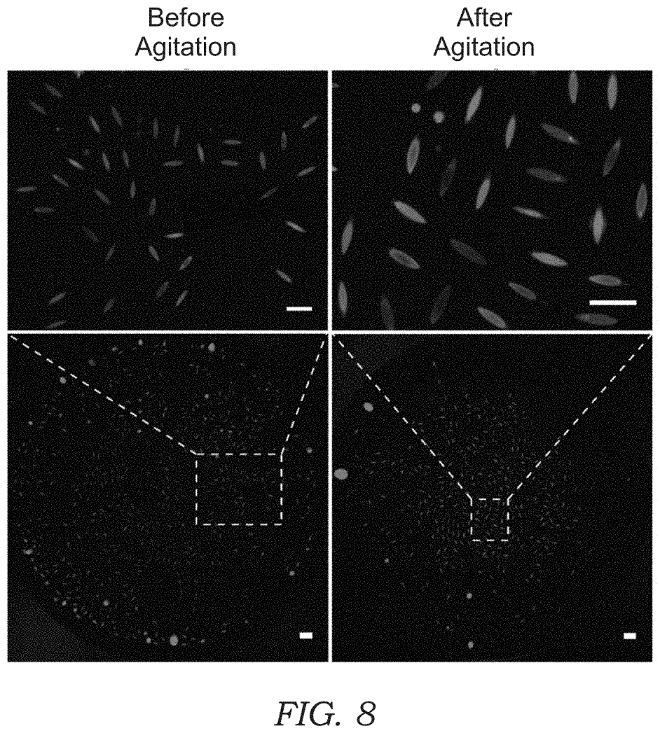

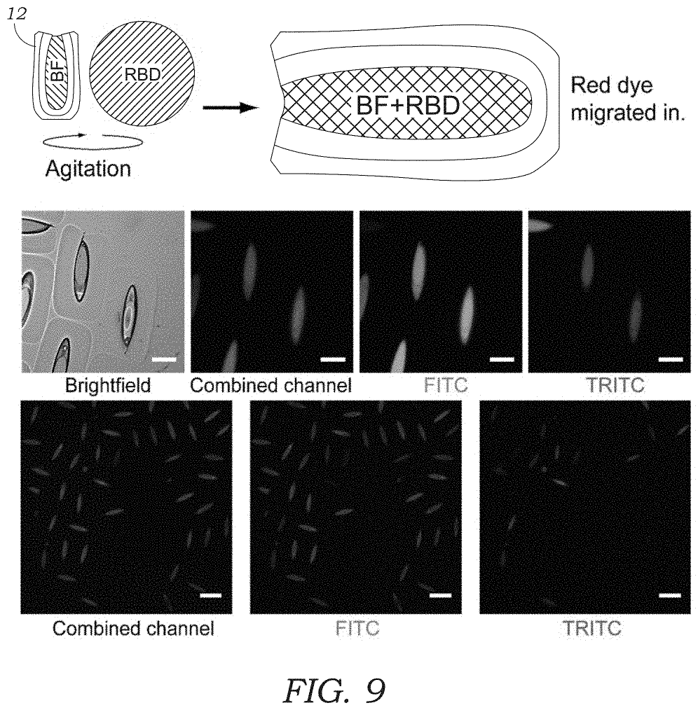

Solution exchange. Unlike surfactants which can facilitate transport of dyes and other molecules out of drops, the armored emulsion system inhibits the transport between the dispersed phase fluid compartments (i.e., the aqueous fluid droplet 18 contained inside the drop-carrier particle 12). Two populations of particle-drops 20 were mixed containing separate dye solutions. Following agitation, the solutions remain in the two respective populations of particle-drops 20 without exchange (FIG. 2D, FIG. 8). Stability to mixing may result from the outer hydrophobic layer or region 16 yielding a physical barrier along with the thermodynamic stability of the supported drops. In contrast, transport into particle-drops 20 can be facilitated by the introduction of larger free drops followed by agitation (FIG. 9). Fluid exchange is enabled through merging and pinch-off events between particle-drops 20 and free drops during mixing.

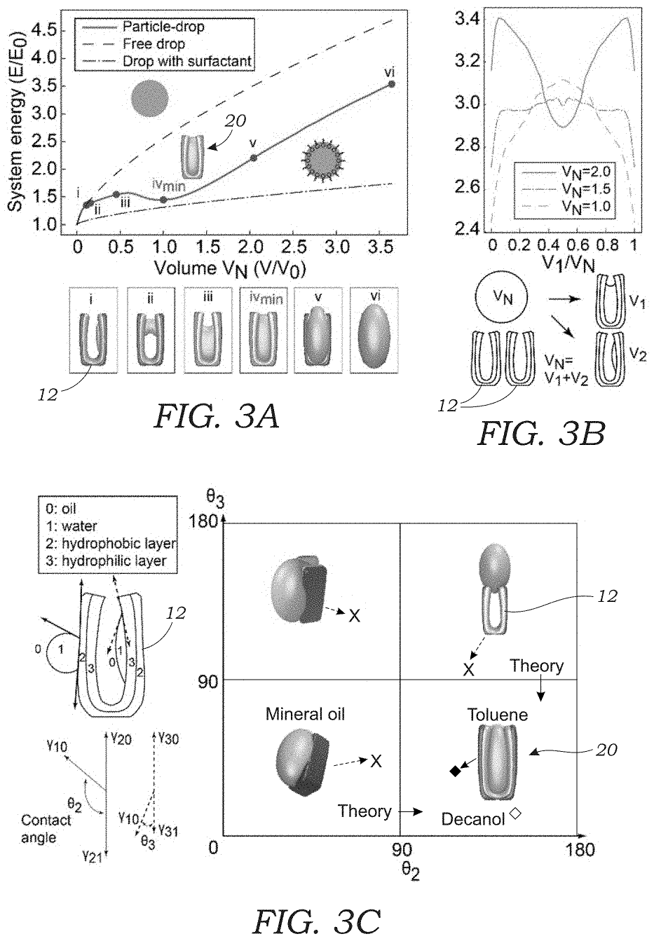

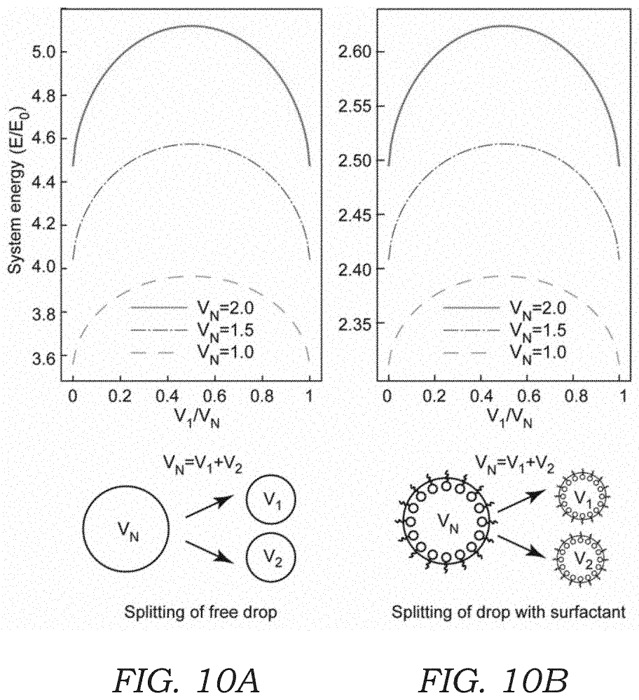

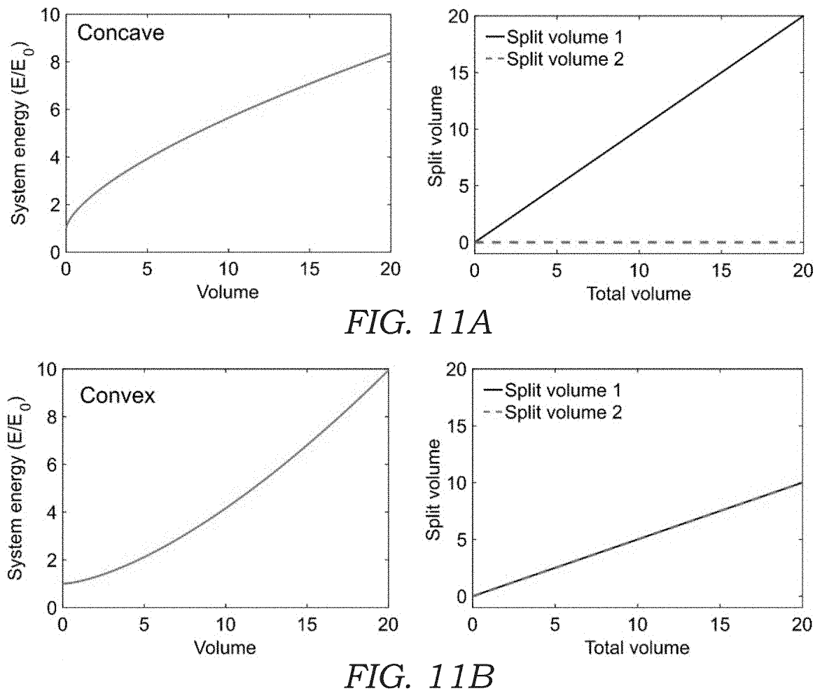

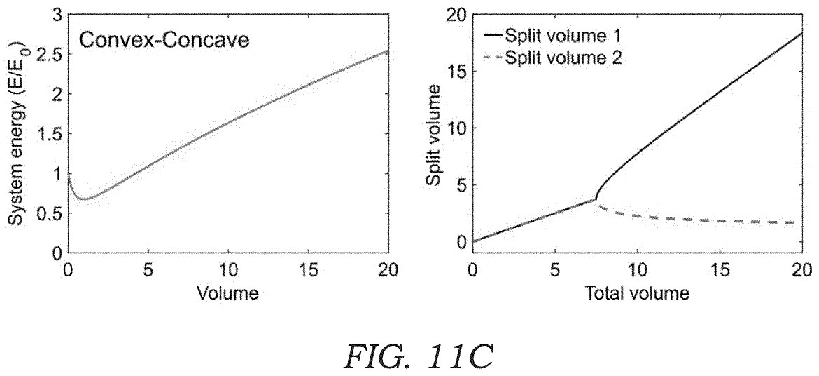

To understand the physics of the particle-drop 30 system, theory was developed based on numerical simulations. In a two-phase system, the interfacial energy increases linearly with surface area; for an isolated sphere of volume (V=4πr³/3), the energy scales as 4πr²˜V^{(2/3)} (FIG. 3A). For spherical drop emulsions, there is no local minimum in drop size and coalescence of adjacent drops is spontaneous due to the overall decrease in surface area (FIG. 10 and FIG. 11A). If the volume vs. interfacial energy (V-E) relationship is instead convex (FIG. 11B), it is energetically favorable for a drop to split into equal volumes. This process of splitting will continue ad infinitum, again leading to no local minimum in drop size. However, if a V-E curve transitions from convex to concave (FIG. 11C), a drop splitting into two daughter drops is expected to break evenly for smaller volumes and break symmetry for larger volumes, with one holding a preferred volume close to the inflection point in the V-E curve, and the other containing the remaining volume (FIG. 11C). For an overall fluid volume exceeding the number of drops multiplied by the preferred drop volume for each drop, this process of asymmetric splitting is expected to accumulate drops with the preferred volume.

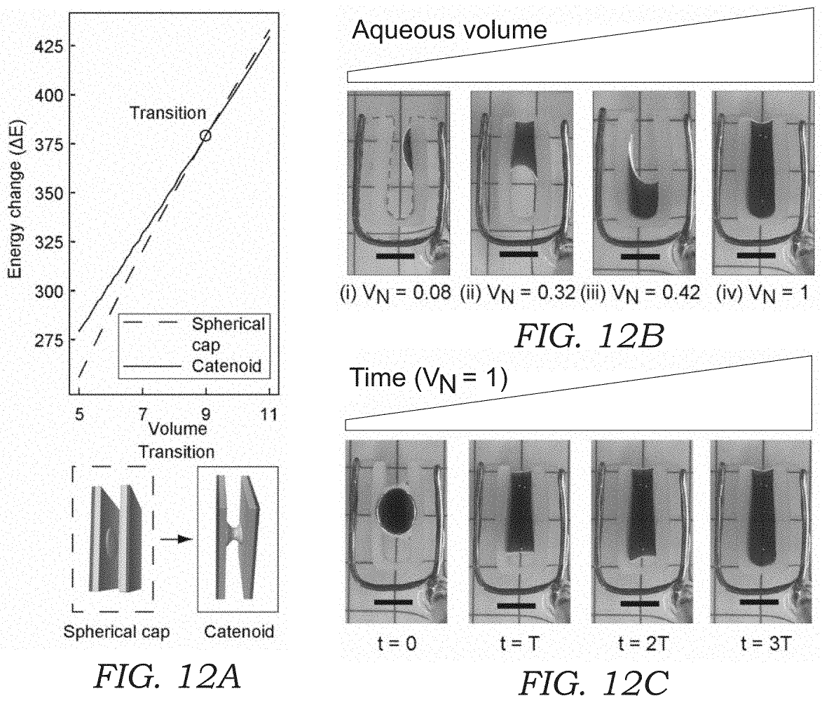

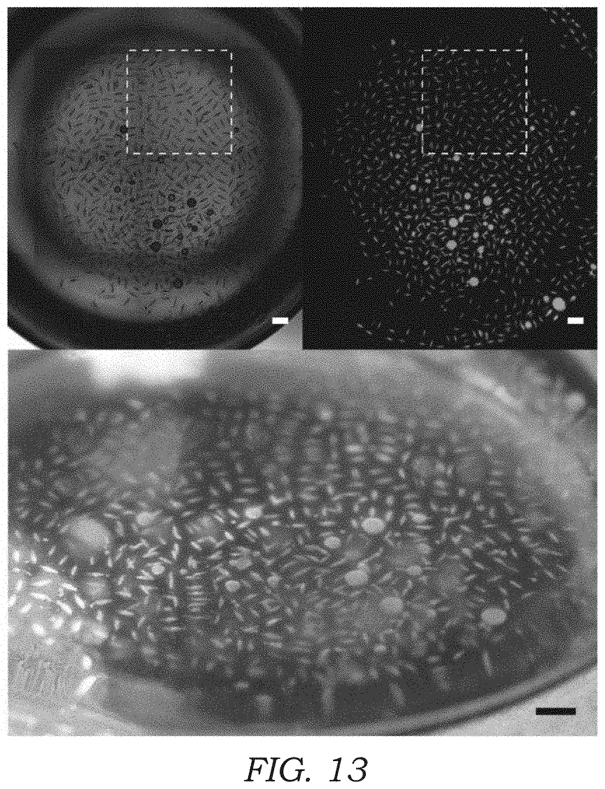

It is hypothesized that such a convex-concave functional form is achievable using microstructures at the length scale commensurate with the desired drop size. Practically, an initial concave region of the V-E curve is expected for small volumes as a small drop behaves as a spherical cap on a surface until it achieves dimensions commensurate with the confining microstructure. This “spreading” phase at low volume, in which increasing volume is accompanied by a decreasing rate of increase in surface energy (concave energy), sets the stage for an “inflationary” phase (convex energy) wherein interfacial energy increases more rapidly with increasing volume as the drop fills the microstructure dimensions. Finally, at larger volumes, the V-E curve returns to a concave form consistent with the behavior of a free drop. These conditions are not met with simple topologies such as drops interacting with planes or parallel plates (FIG. 12A), indicating additional confining surfaces are required, with a trade-off that increasing confinement inhibits drop loading. The design of DCPs 12 therefore balances this trade-off of providing enough wetting surfaces to lower interfacial energy with volume and providing enough access for drop 18 filling within the DCP 12. In one embodiment, this trade-off was satisfied using elongated C-shaped drop-carrier particles 12 with a narrow opening (O) extruded along the orthogonal direction (FIGS. 1A and 1B). Aqueous fluid can access the internal region along the open faces and through the narrow opening (O) at the entry of the C-shape.

DCPs 12 create unique energy minima in the V-E relationship leading to thermodynamic stabilization of drops of specified volumes (FIG. 3). Drop-carrier particles 12 as shown in FIG. 1 were simulated using a volume-constrained minimal surface algorithm for the four phases. The method is an MBO scheme with auction dynamics for the volume constraint. The numerical model indicates an initial spreading phase as a low volume of the dispersed fluid forms a single spherical cap (FIG. 3A, location i). A reduced slope in the V-E curve corresponds to the formation of a bridging catenoid (FIG. 3A, locations ii-iii). As the drop interacts with more than two surfaces for intermediate volumes a local maximum is observed (FIG. 3A, locations iii-iv). Once the interior volume is filled, an inflationary phase is observed in which energy increases with volume at an enhanced rate (FIG. 3A, location v). At even larger volumes, the behavior approaches the asymptotic condition of a spherical drop (FIG. 3A location vi). The volume-dependent final templated-drop morphologies were observed experimentally using a scaled-up system which revealed time-dependent merging of aqueous volumes with the DCP and spontaneous evolution to the minimum energy configuration (FIGS. 12B, 12C).

The shape of this V-E curve results in thermodynamic stabilization of microdrops 18 within DCPs 12, preventing coalescence (FIG. 3B). The energy required to split a drop associated with two DCPs 12 was calculated using the V-E curve. For a supported drop with volume corresponding to the local minimum in energy, V₀, splitting is energetically unfavorable (FIG. 3B, dash line). However, as the overall volume increases to 2 V₀, a global minimum appears when each particle supports a volume of V₀. This mechanism is responsible for creating monodisperse drops whereby an optimal volume of disperse phase, NV₀, is split evenly amongst N particles, with an equilibrium condition of N particles each carrying volume V₀.

The model also provides information on the contact angles that support stable drops. A phase diagram shows the morphology of droplet adhesion to the drop-carrier particle 12 for different contact angles for the hydrophobic and hydrophilic surfaces (FIG. 3C). In the lower right region of FIG. 3C, the drop covers the hydrophilic layer fully without wetting the hydrophobic layer, a condition that experimentally yields monodisperse drops. Conversely for domains outside of this region the drop partially wets the outside layer, leading to unsupported drops. Additional considerations for practical design of DCPs 12 are also necessary.

The ability to create monodisperse emulsions supported by a solid-phase opens up many new opportunities for molecular and cellular assays. As one example, protocols were developed for enzymatic reaction, encapsulation, and cell lysis using particle-drops 20 suspended in PSDS due to its compatibility with chemistry, microalgae cells, and cellular assays. Performing enzyme reaction in the particle-drops 20 enables development of biological assays, such as enzyme-linked immunosorbent assays (ELISA) with high sensitivity. First, the signal from enzyme reactions can be accumulated in the particle-drops 20. Particle-drops 20 were generated with an aqueous solution including enzyme and fluorogenic enzyme substrate (Streptavidin-β-Galactosidase, SβG, and Fluorescein di(β-D-galactopyranoside), FDG). The enzyme has slow activity when quenched at 4° Celsius and then once bringing the system to room temperature the cleavage of FDG to fluorescein was monitored. Showing the cleavage and accumulation of the fluorescein signal over time in this system a bright fluorescent particle-drop 20 was observed (FIG. 4A) after incubation.

Solid-phase enzymatic reactions in the particle-drops 20 were also demonstrated (FIG. 4B). In this experiment, the internal PEG layer of the drop-carrier particle 12 was modified with biotin during fabrication by including biotin-PEG-acrylate. These particles 12 were then bound to streptavidin-labeled horse radish peroxidase (HRP) with various concentrations (1 pM to 5 nM), washed, and particle-drops 20 were generated with aqueous QuantaRed reagent (described in methods herein). After generation of particle-drops 20, the system was incubated for 30 minutes, the particle-drops 20 were imaged under fluorescence microscopy in the TRITC channel (using green excitation light and filtering out red emissions), and the average fluorescent intensity for each concentration of HRP was measured. The results show that HRP with higher concentrations on the inner layers of the DCPs 12 catalyzed the reaction rapidly to generate high concentrations of red fluorescent resorufin, which was isolated in each particle-drop (FIG. 4B). It was also observed that the red fluorescent resorufin can partition at higher concentrations in the inner PEG layer of the DCP 12, leading to enhanced signal to background compared to in the aqueous solution alone, and higher sensitivity detection.

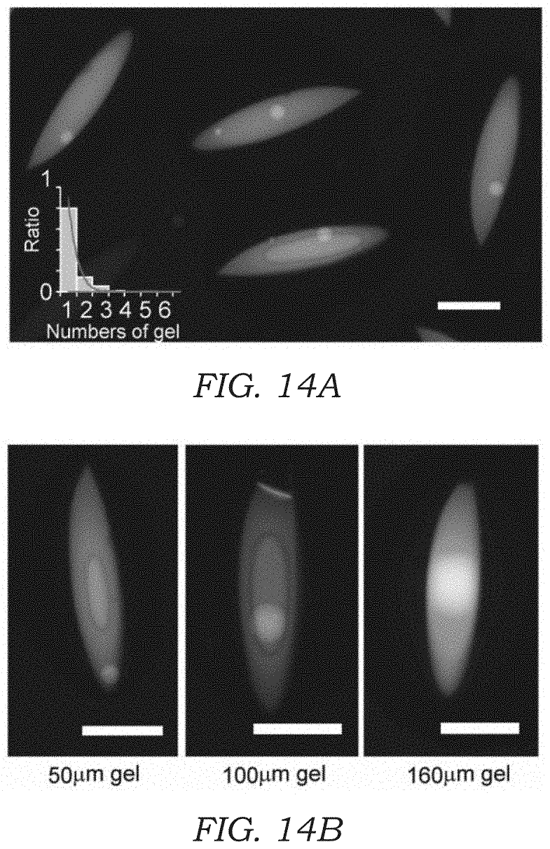

Another application for particle-drops 20 is the analysis of single cells and particles. Experiments were conducted to show the viability of microalgae cells (Euglena gracilis) in the particle-drops 20 using a continuous phase of PSDS 22 over two days. The particle-drops 20 were generated with Euglena in media and incubated for two days (see methods herein for details). After incubation, the Euglena isolated in each particle-drop 20 remained motile with fluorescent signals corresponding to intact chlorophyll pigments at a normal level (FIG. 4C). Moreover, particle-drops 20 were used to encapsulate spherical microgels suspended in a dispersed aqueous phase by following the protocol to form particle-drops 20 in toluene (FIG. 14A) to demonstrate the capability to encapsulate single entities or objects. The number of microgels per drop-carrier particle 12 generally follows Poisson statistics (FIG. 14A inset), and is independent of the gel diameter, as long as the diameter is less than the maximum gap or opening (O) of the cavity 24 within the DCPs 12 (FIG. 14B). Unlike standard emulsions, the DCP 12 can be sized to isolate individual cells or other microparticles, while excluding additional ones.

A protocol to perform cell lysis in the particle-drops 20 was developed after encapsulating cells in the particle-drops 12 (see methods for details below). Particle-drops 20 were generated with Jurkat-cell-laden solutions and particle-drops 20 were allowed to settle on a PDMS surface. The PSDS continuous phase was swapped with PSDS containing methanol and lysis reagents (sodium lauroyl sarcosinate or sarkosyl) time-lapse images were taken to record the process of cell lysis in a particle-drop 20. The result shows that green fluorescent calcein stained Jurkat cells released dye into the particle-drop 20 after the continuous phase 22 was swapped (FIG. 4D), indicating poration of the membrane and lysis. Cell lysis can thus be achieved in the particle-drops 20 by selective molecular exchange between continuous and dispersed phases, which presents a unique property for cell manipulation using particle-drops 20, which is less easily achieved with surfactant stabilized drops. The ability to exchange reagents through the continuous phase 22 that can then partition into the aqueous phase 18 of the particle-drops 20 can be used for other applications in performing bioassays, such as providing fluorogenic substrates for enzymatic reactions.

In order to achieve reagent exchange through the oil phase 22 the reagent that is desired to exchange was found to be miscible in both the surrounding oil continuous phase 22 and disperse aqueous phase present in the interior of the particle-drops 20 as droplets 18. Preferably, the miscibility in the aqueous phase is higher than in the continuous surrounding phase such that the reagent accumulates in the particle-drops 20 at a higher concentration. If the reagent is consumed in a reaction in the particle-drop 20 (e.g., for a fluorogenic substrate converted to a fluorophore, or a surfactant inserting into cell membranes) this can drive continued partitioning of the reagent into the particle-drop 20 from a large reservoir of reagent in the surrounding oil phase 22. This is important to transfer high quantities of reagent into the particle-drop 20 even if the saturation miscibility of the continuous oil phase 22 may be at low concentrations.

Materials and Methods:

Auction Dynamics Simulations

Droplet Encapsulation Simulation Preparation. A triangulated mesh defining the hydrophobic and hydrophilic surfaces of the drop-carrier particle is used. This is mapped to a 3D Cartesian grid in which the Cartesian grids are classified into one of four categories: hydrophobic, hydrophilic, droplet, or oil domain. To achieve this, the improved parity algorithm developed for an Eulerian solvent excluded surface is applied as described in Liu, B., Wang, B., Zhao, R., Tong, Y. & Wei, G.-W. ESES: Software for Eulerian solvent excluded surface. J. Comput. Chem. 38, 446-466 (2017), which is incorporated by reference. For a given point x, one draws a half-line emanating from x and count how often it crosses the triangles. The number of crosses determines the phase in which x is located in.

Droplet Encapsulation Simulation. In the microscale particle droplet system, the dominant interaction comes from the surface tension between different phases. By ignoring the other forces, one solves for a minimum surface energy configuration using the Auction Dynamics algorithm on the Cartesian grid. Auction dynamics generates a discrete timestep approximation of volume preserving mean curvature motion of the interfacial boundaries between phases, preserving the volumes of all the phases. As a result, configurations that are stationary under the flow are surface energy minimizers, which is iterated from an initially spherical droplet on top of the capsule.

Droplet Encapsulation System Post-processing. The contact area of each pair of phases is computed to further compute the surface energies of the energy minimization configuration. To systematically address this issue, one first smooths the initial non-smooth sharp interface by running a few steps of Laplacian smoothing. Then the marching cubes algorithm is applied to extract the level set from the smeared interface. Finally, one triangulates the extracted level set by using the CGAL software and compute its contact area straightforwardly.

Design Considerations for Drop-Carrier Particles (DCPs)

There are additional considerations for practical design of DCPs 12. For example, drop-carrier particles 12 should be largely closed such that multi-particle supported drops are energetically unfavorable and monodispersity is preserved (FIGS. 11A and 11B). In addition, the model assumes that interfacial energies will dominate the behavior of the system, which is valid when factors such as buoyancy remain small. The Bond number, Bo=Δμgd²/Δσ, for the experimental system is ˜4×10⁻⁴, reinforcing this assumption. Here, Δρ is the density difference between the disperse and continuous phase, g is acceleration due to gravity, d is the width of the interior void of the drop-carrier particle, and Δσ is the difference between the interfacial tension of the disperse phase and continuous phase with the interfacial tension between the disperse phase and hydrophilic internal material. The interfacial tension of the outer hydrophobic material with the continuous phase should also be small compared to thermal energy to prevent aggregation of drop-carrier particles 12 due to favorable particle-particle contacts on their outer surfaces.

Manufacture of Drop-Carrier Particles

High-throughput optical Transient Liquid Molding for manufacturing of drop-carrier microparticles. Because there are no commercially available microparticles with complex 3D shapes comprising materials with separate wettability, and no standard methods to fabricate such particles, optical Transient Liquid Molding (OTLM) was used to photocrosslink precursor streams into the shape of drop-carrier particles 12, as shown in FIG. 5. OTLM integrates inertial flow engineering, flow control, and photolithography to fabricate drop-carrier particles 12 with complex shapes, compositions, and surface properties in 3D. First, a co-flow of polymer precursors is pumped into a microfluidic channel 32 with a designed sequence of micropillars 30 at a Reynolds number of 5 to 40. Fluid inertia of the flow around the micropillars 30 leads to an irreversible deformation of a starting rectangular co-flow pattern to a complex cross-sectional pattern. A sequence of micropillars 30 with various sizes and lateral positions can be used to design a wide diversity of cross-sectional patterns, including concave, convex, diamond, stretched bars, etc. Once a pattern is developed downstream of the microfluidic channel 32 containing the micropillars 30, the flow is rapidly stopped and pressure equalized in the microfluidic channel 32 by simultaneously stopping the upstream pump 40 and occluding the outlet tubing downstream with a pinch valve 36. Within 1 second, the sculpted precursor stream is then illuminated with patterned UV light for 500 ms to photocrosslink the precursor stream and solidify multiple 3D-shaped particles 12. The patterned UV light is created by coupling collimated UV light 38 to a chrome mask 34 with an array of transparent rectangles (140×600 μm). Following photocrosslinking, the downstream pinch valve 36 is re-opened and the pump 40 is again re-started to flush cured drop-carrier particles 12 into a container 44 outside of the microfluidic channel 32 and re-develop the precursor flow stream for the next UV illumination cycle. This manufacturing cycle is automated using LabVIEW in order to fabricate large batches of particles. The reproducibility of particle shape across a population of the particles 12 was investigated and previously have shown that within ˜20 cm downstream, the difference between the precursor flow shape in simulation as well as the particles fabricated along the flow length is negligible due to the high Peclet number of the flow.

Microfluidic channel design. The drop-carrier particles 12 were designed using custom software built in lab and open to the public, called μFlow (described herein). μFlow enables rapid computation of a 3D particle shape formed from the intersection of an extrusion of the flow stream cross-sectional shape and an extrusion of an orthogonal 2D optical mask shape. Real-time design of the particle shape is possible since the advection maps associated with the inertial flow around a pre-simulated library of pillars 30 is stored and the flow deformation from a pillar sequence is rapidly computed without fluid dynamic simulations. Six micropillars 30 adjacent to the wall of the microfluidic channel 32 can generate a cross-sectional flow pattern with concentric layers with only a small opening on one side, which is suitable for drop-carrier particles 12 when patterned with a rectangular optical mask 34 (see inset of “cross section of co-flow” in FIG. 5).

Microfluidic chip fabrication. Microfluidic chips containing sequences of pillars 30 designed to create the cross-sectional pattern with concentric layers of the precursor flow stream were fabricated using conventional soft lithography. The microchannel 32 also contained a long downstream region after the pillars 30 to expose a linear array of patterns to increase fabrication throughput. The silicon mold for replicating poly(dimethylsiloxane) PDMS channels was designed to be 300 μm in thickness and so required a specialized process. First, a layer of SU-8 2100 (MicroChem Corp.) was spun to a thickness of 200 μm onto a wafer, recovered thermal stress, and a second layer of SU-8 with 100 μm thickness was spun. Then, following standard protocols for photolithography, the mold was developed. Then, PDMS (Sylgard 184, Dow Corning) was cured on top of the mold to replicate the microchannel 32, peeled the PDMS device off the wafer, punched holes for inlets and an outlet, and bonded to a glass slide coated with a thin layer of PDMS using air plasma. The thin PDMS layer was required to match the surface wetting properties across all walls of the microchannel 32. The PDMS precursor was spun on the slide at 1000 rpm for 30 seconds and cured in an oven overnight.

Polymer precursor preparation. Poly(ethylene glycol) diacrylate (PEGDA, M_{w}≈575; 437441, Sigma-Aldrich) and poly(propylene glycol) diacrylate (PPGDA, M_{w}≈800; 455024, Sigma-Aldrich) were chosen to be the polymer precursors for the hydrophilic region 14 and hydrophobic region 16 of the drop-carrier particles 12, respectively. These materials satisfied interfacial tension conditions of importance as described in FIG. 3C and compatibility with the OTLM process. When multiple precursor fluids are employed in OTLM, the precursors should be miscible with each other to avoid the effect of finite interfacial tension at the interface of the co-flow which would act against the deformation generated by flow inertia. Moreover, to eliminate the asymmetry created by the density difference between co-flowing streams which can lead to differential settling over a finite flow stopping time, PEGDA and PPGDA were diluted to 60% and 90% with ethanol respectively so the density of all liquids was matched at 0.987 g/mL. The concentration of the photoinitiator (2-hydroxy-2-methylpropiophenone, Darocur 1173, 405655, Sigma-Aldrich) in the two precursors was tuned in the experiment until the speed of the photocrosslinking was equalized and the particles can be manufactured such that both polymers are cured. The concentration was 1.3% and 2.6% in diluted PEGDA and PPGDA respectively. After fabrication, all drop-carrier particles 12 were collected in a 50 mL centrifuge tube and rinsed with a volume of ethanol more than 1000 times the sample volume to eliminate non-crosslinked reagents. The drop-carrier particles 12 were stored in ethanol for later usage.

Protocol for Particle-Drop Generation

Toluene as continuous phase. To reduce the numbers of particle-free satellite drops and adhesion between drop-carrier particles 12 and the glass container, a mix of toluene with 10-15% ethanol was used. The protocol to create particle-drops 20 is as follows: (1) disperse drop-carrier particles 12 (initially in ethanol) in 1 mL of the toluene/ethanol mix, (2) inject aqueous solution typically ˜20 μL (˜17 times of the total void volume of particles), (3) pipette the solutions vigorously in a 20 mL scintillation glass vial (VWR, LLC.) with a hydrophobic coating which is introduced by incubation with Rain-X® (ITW Global Brands) for 2 days, (4) centrifuge down the solution in the vial at 2000 rpm for 5 minutes at 25° C., and (5) pipette away visible satellite drops, cover the vial with parafilm for storage. It was confirmed that the particle-drops 20 can be generated with the same morphology in another liquid system composed of toluene/water and toluene/water/surfactant (Pluronic F-127, Sigma-Aldrich) without ethanol. In this protocol ethanol was evaporated and the drop-carrier particles 12 with a large amount of water with 1-4% (w/v) Pluronic to disperse particles 12 in an aqueous instead of organic phase. The drop-carrier particles 12 in aqueous solution were then mixed with toluene and centrifuged as described above.

Poly(dimethylsiloxane-co-diphenylsiloxane) (PSDS) as the continuous phase. DCPs 12 were rinsed with 1 mL 0.5% Pluronic in PBS three times and then the DCPs 12 were dispersed in PBS with Pluronic in a 20 mL scintillation vial and the DCPs 12 were allowed to settle. The supernatant was removed until the volume of solution was 100 pt. 1 mL PSDS was dispensed into the vial and pipetted up and down twice without generating bubbles to generate suspended particle-drops 20. The mixed PSDS emulsion with particle-drops 20 was transferred into a new scintillation vial with Rain-X® treatment. Because the DCPs 12 have significantly higher density than the oil continuous phase 22 this allows for easy isolation and accumulation of particle-drops 20 at the bottom of the vial due to gravitational forces. The higher density of particle-drops 20 and shape which is narrower in cross-section than across the face holding the aqueous drop 18 also allows the particle-drops 20 to settle with the majority of particle-drops 20 in the same orientation (e.g., showing a C-shaped face when imaged from the bottom of the vial).

Imaging and image processing. The particle-drops 20 were imaged using fluorescence microscopy. For clear visualization, 100 μg/mL biotin-4-fluorescein (BF, Catalog number: 50849911, Fisher Scientific) was added into the aqueous solution. A custom Python code was used to analyze the images of particle-drops 20. The code detected the fluorescent regions representing drops 18, filtered out regions with size larger than twice or smaller than 0.375 times the nominal size of the particle 12 which corresponded to satellite drops not associated with particles 12. The size/circularity/total intensity were measured for targets, and exported an image after filtering, and compared it to the brightfield image for confirmation. For the study of long-term stability, the image was also filtered using circularity to ensure only particle-drops 20 were investigated without considering satellite drops.

Method of Enzymatic Reaction in Solution Inside of Particle-Drops

A solution-phase enzyme reaction was demonstrated using β-galactosidase. DCPs 12 were fabricated and then dispersed in DPBS with 0.5% w/v Pluronic. 100 μg/mL streptavidin-β-galactosidase (SβG, Sigma-Aldrich), 100 μg/mL fluorescein di(β-D-galactopyranoside) (FDG, Sigma-Aldrich), and DCPs were pre-mixed at 4° Celsius to quench the reaction during liquid handling. Poly(dimethylsiloxane-co-diphenylsiloxane) (PSDS, Sigma-Aldrich) as the continuous phase 22 and pipetted the PSDS and pre-mixed solution up and down 2-3 times to generate particle-drops 20 in a glass vial. The vial was brought back to room temperature to initiate the enzyme reaction. The brightfield and fluorescent images of a particle-drop 20 were taken before and after overnight incubation, to observe the reaction at completion.

To demonstrate enzymatic reactions that can accumulate product in particle-drops 20 for enzymes immobilized on the solid-phase (i.e., the drop-carrier particle 12), additional steps were incorporated into the protocols for drop-carrier particle 12 manufacture and particle-drop 20 generation. First, DCPs 12 were fabricated to contain a biotinylated inner PEG layer 14 to be suitable for downstream functionalization of the DCPs 12 through biotin streptavidin linkages or other linkages to biotin. In the fabrication step, a mix of PEGDA, ethanol, biotin-PEG-acrylate (Catalog number: PG2-ARBN-5k, NANOCS) in DMSO was used as the precursor of the inner layer for simultaneously grafting biotin within the PEG layer 14 during photocrosslinking. After fabrication, the drop-carrier particles 12 were rinsed. Horse radish peroxidase (HRP) enzyme was then bound to the DCP 12 surface as a model of an affinity reaction, like an immunoassay. A solution of Streptavidin-conjugated HRP (Catalog number: N100 Thermo Fisher Scientific) was incubated for 30 minutes with DCPs 12 in buffer, the supernatant was removed, and the drop-carrier particles 12 were rinsed with 1 mL PBS with 0.5% Pluronic 10 times to remove all unbound HRP. The drop-carrier particle 12 solution and QuantaRed (Catalog number: 15159, Thermo Fisher Scientific) reagent with a 10:1 ratio of QuantaRed solution to drop-carrier particle solution. The QuantaRed reagent is comprised of 50 parts stable peroxide solution, 50 parts enhancer solution, and 1-part ADHP concentrate following vendor instructions. 1 mL PSDS was dispensed into the combined solution to form particle-drops 20 containing the QuantaRed™ reagent and left the armored emulsion to incubate in a vial with Rain-X® treatment for 30 minutes. Fluorescence microscopy was used to image the product (resorufin) of the enzymatic reaction using green excitation light and red filtered emission light (TRITC filter set).

Method of Microalgae Viability Characterization Inside of Particle-Drops

A microalgae solution comprising Euglena gracilis cultured in KH media was mixed with a drop-carrier particle 12 solution in PBS with 0.5% Pluronic in a vial. PSDS was dispensed into the vial and then the mixture was pipetted 2-3 times to generate particle-drops 20 encapsulating Euglena. The mix was left in a vial with Rain-X® treatment for 2 days and then the viability of the encapsulated Euglena was checked using microscopy by evaluating motility and chlorophyll fluorescence.

Method of Cell Lysis Inside of Particle-Drops

Jurkat cells were stained with calcein to evaluate the process of cell lysis in particle-drops. The Jurkat-cell-laden solution was mixed with drop-carrier particle 12 solution in PBS with 0.5% Pluronic in a vial. PSDS was dispensed into the vial and then the mix was pipetted 2-3 times to generate particle-drops 20 encapsulating Jurkat cells. The mix was left in a PDMS well for ˜30 minutes to allow particle-drops 20 to settle to the bottom of the well in the PSDS continuous phase. New PSDS was then added containing 10% methanol saturated with sarkosyl detergent. Time-lapse images in the FITC channel were taken to monitor the partitioning of the lytic elements through the oil into the particle-drops 20 leading to release of calcein dye from the lysing cells and accumulation in the isolated particle-drop within <10 min.

Microgel Manufacture and Encapsulation Inside of Particle-Drops

Flow focusing device fabrication. A PDMS flow focusing droplet generator device was fabricated using standard soft lithography techniques as described above with a few modifications. KMPR 1010 and 1050 (MicroChem) were used in place of SU8 2100 to fabricate channel molds with heights of 18 μm and 70 μm, respectively. The PDMS device was bonded directly to a glass microscope slide after air plasma activation and then modified with Aquapel™ to render the microfluidic channel 32 surfaces fluorophilic.

μGel Fabrication. Hydrogel microparticles (μGels) used for the encapsulation studies were fabricated using the flow focusing droplet generator device. A gel precursor solution composed of 10 wt % 4-arm PEG-VS (20 kDa) (NOF) in 300 mM triethanolamine (Sigma-Aldrich), pH 8.25 and a crosslinker solution composed of 8 mM dithiothreitol crosslinker (Sigma-Aldrich) solution in DI water pre-reacted with 30 μM Alexa Fluor 568 maleimide (Invitrogen) were co-flowed into the flow focusing device with equal flow rates. The injected solutions segmented into droplets with a pinching continuous phase composed of Novec 7500 oil (3M) and 0.5% Pico-Surf™ (Sphere Fluidics) as a surfactant. The channel dimensions and flow rates used to fabricate the different sized μgels are shown in Table 1.

TABLE 1

| | | | Total | Oil |
|---|---|---|---|---|
| μGel | Channel | Channel | Aqueous | flow |
| Size | Height | Width | flow rate | rate |
| (μm) | (μm) | (μm) | (μL/min) | (μL/min) |
| 50 | 18 | 35 | 4 | 8 |
| 100 | 70 | 65 | 10 | 30 |
| 160 | 70 | 65 | 5 | 1.5 |

μGel precursor droplets were collected in an Eppendorf tube and allowed to crosslink at room temperature overnight. Crosslinked μGels were extracted from the oil using a series of washing steps. Excess oil was removed by pipetting and a solution of 20 wt % perfluorooctanol (Sigma-Aldrich) in Novec 7500 oil to wash away surfactant. DI water was added to swell and disperse the gels. The remaining Novec 7500 oil was removed by addition of hexane to lower the density of the fluorinated oil. μGels were pelleted using a table top centrifuge at 2000×g for 5 min and Novec 7500+hexane supernatant was removed by pipetting.

Protocol of microgel encapsulation in particle-drops. μGels were dispersed into a solution of PBS with 0.1% Pluronic-F127 and 100 μg/mL BF at a concentration of ˜20 μGel/μL. 20 μL of a μGel-laden solution was mixed with drop-carrier particles 12 dispersed in toluene-ethanol mix and then centrifuged down to generate particle-drops 20 encapsulating μGels. The particle-drops 20 and μGels were then imaged in FITC and TRITC channels respectively and combined two images to determine the numbers of gels per encapsulation.

While embodiments of the present invention have been shown and described, various modifications may be made without departing from the scope of the present invention. The invention, therefore, should not be limited except to the following claims and their equivalents.

1. An emulsion system comprising:
a plurality of monodisperse particle-drops wherein each particle-drop is formed by a single elongated drop-carrier particle disposed in an oil-based continuous phase, wherein the single elongated drop-carrier particle comprises an elongate body with an opening at one end thereof, the single elongated drop-carrier particle having a hydrophilic interior region containing an aqueous droplet and a hydrophobic exterior region.

2. The emulsion system of claim 1, further comprising a plurality of satellite drops devoid of any drop-carrier particles contained therein.

3. The emulsion system of claim 1, wherein the emulsion system containing the plurality of monodisperse particle-drops is substantially free of satellite drops devoid of any drop-carrier particles contained therein.

4. The emulsion system of claim 1, wherein the oil-based continuous phase comprises poly(dimethylsiloxane-co-diphenylsiloxane) (PSDS).

5. The emulsion system of claim 1, wherein the oil-based continuous phase comprises toluene or decanol or a mixture thereof.

6. The emulsion system of claim 1, wherein the single elongated drop-carrier particle comprises a C-shaped particle.

7. The emulsion system of claim 1, wherein the opening spans a distance of less than 100 μm.

8. The emulsion system of claim 1, wherein the opening spans a distance of is less than 50 μm.

9. The emulsion system of claim 1, wherein the hydrophilic interior region comprises an enzyme linked directly or indirectly to a surface thereof.

10. The emulsion system of claim 1, wherein the hydrophilic interior region comprises a fluorophore or label linked directly or indirectly to a surface thereof.

11. The emulsion system of claim 1, wherein the hydrophilic interior region comprises one or more reagents, analytes, labels, reporter molecules, beads, and/or cells immobilized to a surface thereof.

12. The emulsion system of claim 1, wherein the aqueous droplet contains one or more reagents, analytes, labels, reporter molecules, beads, and/or cells.

13. The emulsion system of claim 1, wherein the hydrophilic interior region comprises a poly(ethylene glycol)-based material.

14. The emulsion system of claim 1, further comprising a single live cell contained in one or more of the plurality of monodisperse particle-drops.

15. The emulsion system of claim 1, wherein the plurality of monodisperse particle-drops maintain their monodisperse characteristics for several days.

16. The emulsion system of claim 1, wherein the hydrophilic interior region comprises a porous material and wherein the porous material contains a reaction product therein for an extended period of time.

17. The emulsion system of claim 1, wherein the hydrophilic interior region comprises a porous material and wherein the porous material contains a fluorophore or other light emitting molecule therein for an extended period of time.

18. The emulsion system of claim 14, further comprising a surfactant or detergent added to the oil-based continuous phase.

19-30. (canceled)

31. An emulsion system comprising:
a plurality of monodisperse particle-drops wherein each particle-drop is formed by a single elongated solid-phase particle disposed in an oil-based continuous phase, wherein the single elongated solid-phase particle comprises an elongate body with an opening at one end thereof, the single elongated solid-phase particle having a hydrophilic interior region containing an aqueous droplet and a hydrophobic exterior region, wherein the solid-phase particle and/or the aqueous droplet contains one or more reagents, analytes, labels, reporter molecules, and/or cells therein.

32. The system of claim 31, wherein the one or more reagents, analytes, labels, reporter molecules, and/or cells are immobilized to the hydrophilic interior region of the solid-phase particle.
